(19) United States
(12) Patent Application Publication
(10) Pub. No.: US 2019/0053962 A1
(43) Pub. Date: Feb. 21, 2019

(54) Umbilical Cord Cover

(71) Applicant: Gary Dean Lavon, Liberty Township, OH (US)

(72) Inventor: Gary Dean Lavon, Liberty Township, OH (US)

(21) Appl. No.: 16/104,122

(22) Filed: Aug. 16, 2018

Related U.S. Application Data
(60) Provisional application No. 62/546,593, filed on Aug. 17, 2017.

(51) Int. Cl. A61F 15/00

(52) U.S. Cl. CPC A61F 15/008

(57) ABSTRACT

A protective umbilical cord cover having a containment portion being adapted to protect an umbilical cord of an infant and also an attachment mechanism being adapted to releasably attach the containment portion against the infant. The protective umbilical cord cover may also include an umbilical cord cover material that is constructed to form the containment portion, wherein the umbilical cord cover material is liquid impermeable in an inward-flow direction. The protective umbilical cord cover may also include an umbilical cord cover material that is constructed to form the containment portion, wherein the umbilical cord cover material is vapor permeable in an outward-flow direction. The protective umbilical cord cover may also include an umbilical cord cover material that is constructed to form the containment portion, wherein the umbilical cord cover material is vapor permeable in an outward-flow direction.

### FIELD OF THE INVENTION

In developing countries, umbilical cord infections contribute significantly to high neonatal mortality rates. Such infections can be reduced by practicing clean delivery and clean cord care practices. Tetanus and infections are among the leading causes of infant mortality. Some 500,000 infants die of neonatal tetanus and an additional 400,000 die as a consequence of severe bacterial infections, a substantial portion of which result from umbilical cord infections. Information from the World Health Organization has shown in one hospital study, conducted in developing countries, that in 47% of infants hospitalized with sepsis, cord infection was the source of the illness. Furthermore, 21% of infants admitted for other reasons had omphalitis, cord infection. Cord infections in developing countries can be prevented through promoting clean umbilical cord care and minimizing exposure of the cord to contaminated objects such as contaminated water and clothing that has been washed in contaminated water. The protective umbilical cord covers of the present invention address all of the concerns and issues previously mentioned while allowing the umbilical cord to heal naturally.

Even in developed countries, where umbilical cord infections are rare, the umbilical cord and its care introduces other issues, such as a significant level of stress and concern of new parents at a time when they should be enjoying the parenting experience. The key points of concern with respect to the umbilical cord are hygiene of the cord, keeping the cord dry and keeping the cord from becoming entangled or adhered to clothing.

The present invention will help reduce the incidence of umbilical cord infections, as well as address these concerns and provide the mother with more flexibility with respect to how she chooses to care for her baby, especially how the baby is bathed.

The invention further relates to an umbilical cord cover which can be joined to an umbilical venous catheter.

### BACKGROUND OF THE INVENTION

Newborn infants have an umbilical cord which is the remnant of the cord-like structure which provided the connection between the fetus and the placenta of the mother in utero. The umbilical cord is exposed on the outside of the abdomen of a newborn infant. The umbilical cord is typically left uncovered in order to allow the cord to dry, heal and eventually to detach from the body creating the “belly button” navel.

During the first few days following birth, the blood vessels in the umbilical cord are still viable and unobstructed thereby providing direct access to the infant's bloodstream. The period of highest risk for umbilical cord containment with bacteria and infection is the first three days of life. The umbilical cord represents a common means of entry for systemic infection into a newborn infant's bloodstream. Such infections may remain localized or spread internally potentially affecting the lungs, pancreas, heart and kidneys. Furthermore, the infection may also spread by direct extension into the peritoneal cavity, leading to peritonitis. Keeping the umbilical cord clean, dry and isolated from contaminants, including bacteria sources, is very important to ensure proper healing and to avoid such infections. The umbilical cord typically remains attached to the umbilicus for approximately two weeks. The umbilical cord requires a great deal of care during this two week period. During the time the umbilical cord is attached, the caregiver is restricted to cautiously giving the infant quick sponge baths versus a more thoroughly cleansing baths, such as can be obtained through submersion, in order to avoid wetting the umbilical cord. Wetting of the umbilical cord can extend the drying time, healing, and can also increase the risk of an infection. Infections can also become problematic even for quick sponge baths if the water being used is not clean and hygienic. This is a particularly common cause of umbilical cord infections in developing countries. Additionally, washing the baby's clothing in unclean water can also lead to an umbilical cord infection as the clothing can directly contact the umbilical cord.

The umbilical cord can also come into contact with feces, especially runny bowel movements that can spread into the front of the diaper while being worn by the infant. Such runny bowel movements are common among newborn infants, especially those that are breastfed. Contact between the umbilical cord and feces can increase the risk of an umbilical cord infection.

The umbilical cord can sometimes bleed, as well as, ooze small amounts of liquid during the drying process. These excretions often cause the cord to adhere to a diaper or surrounding clothing thus making removal of the diaper or clothing difficult. In order to remove, for example, clothing that has become adhered to the umbilical cord without damaging the cord itself, it may become necessary to wet the cord slightly to get it to release. The wetting of the cord to promote this release tends to lengthen the healing, drying, time and may present a source for infection.

The umbilical cord also develops a rough surface as it dries which can result in the cord becoming snagged on clothing as the baby moves or as the mother removes the clothes. The cord becoming entangled with or adhered to the clothing can result in damage to the cord and therefore requires extreme care when changing the infant's clothing. Such damage to the cord can also lengthen the time it takes for the cord to heal.

Infants, particularly premature infants, can exhibit a trans-epidermal water loss, TEWL, of greater than about 100 ml/kg/day, or greater than about 2000 g/m²/day. TEWL measurements of full-term infants can range from about 18 ml/kg/day or about 400 g/m²/day to greater than about 100 ml/kg/day or 2000 g/m²/day. It would therefore be preferable for the protective umbilical cord cover of the present invention to have a Moisture Vapor Transmission Rate, MVTR, equal to or greater than the TEWL of the wearer.

Infants, particularly pre-term infants, are susceptible to epidermal stripping due to an immature epidermis. Epidermal stripping can result from the use of aggressive adhesives on infant skin, such as the adhesives used to affix trans-cutaneous monitors and other monitoring devices. In addition, the use of adhesive tape to affix various devices can result in significant epidermal stripping. Epidermal stripping may increase the risk of infection especially for infants with an immature epidermis. Many alternative approaches have been identified to mitigate these adverse effects. Two such approaches are the use of pectin barriers in conjunction with adhesives and alternatively the use of hydrogel or hydrocolloid adhesives. Low-peel strength adhesives, such as low peel hydrogel or hydrocolloid adhesives can provide adequate adhesion to the skin and minimize or eliminate epidermal stripping due to their low-peel force characteristics.

Therefore, it is the intent of the present invention to provide a protective umbilical cord cover designed to contain and protect the umbilical cord and prevent the cord from becoming wetted or damaged.

It is further the intent of the present invention to provide a protective umbilical cord cover comprising an affixing means such as a low-peel force adhesive such as a hydrogel or hydrocolloid adhesive, or alternatively to provide a protective umbilical cord cover which can be combined with a separate affixing means, such as an extensible belt.

It is further the intent of the present invention to provide a protective umbilical cord cover having a level of vapor transmission adequate to promote proper drying of the umbilical cord while providing the necessary liquid imperviousness to prevent wetting of the cord.

### SUMMARY OF THE INVENTION

The present invention includes a protective umbilical cord cover comprising a containment portion being adapted to protect an umbilical cord of an infant and an attachment mechanism being adapted to releasably attach the containment portion against the infant. The containment portion may comprise a containment shell and the protected air space positioned between the infant and said containment shell. The containment shell may be constructed of a material that is liquid impermeable in an inward-flow direction. The containment shell may be constructed of a material which is also vapor permeable in an outward-flow direction. The phrase “inward-flow direction”, as used herein, means the direction of flow from the outside of the protective cover toward the inside surface and further the inside air space of the protective cover. Conversely, the phrase “outward-flow direction”, as used herein, means the direction of flow from the inside surface and air space of the protective cover toward the outside of the protective cover.

The attachment mechanism may comprise a belt. The belt may be made of an extensible material. The belt may be made of an elastically extensible material.

Alternatively, the attachment mechanism may comprise an adhesive. The present invention contemplates the use of any adhesive safe for use on the skin. In one embodiment, the adhesive may comprise a hydrogel adhesive. In another embodiment, the adhesive may comprise a hydrocolloid adhesive.

In yet another alternative embodiment, the attachment mechanism may comprise a fastening mechanism. Any mechanism of fastening surfaces together is contemplated by this invention. In one embodiment, the fastening mechanism may comprise velcro. In another embodiment, the fastening mechanism may comprise micro-suction.

The protective umbilical cord cover may also comprise a flange portion that substantially surrounds the skin contact area of the protective umbilical cord cover of the containment portion and provides greater surface area for contact with the wearer's skin. The flange portion may provide an underneath surface for an adhesive to be applied. The flange portion may comprise a moisture vapor permeable material. The containment portion may be formed using deformation processes. The containment shell may comprise an extensible material.

The present invention may include a protective umbilical cord cover comprising a containment portion being adapted to protect an umbilical cord of an infant and also an attachment mechanism being adapted to releasably attach the containment portion against the infant. The attachment mechanism may comprise a belt. The containment portion may comprise a containment shell and the protected air space positioned between the infant and said containment shell. The containment shell may be constructed of a material which is liquid impermeable in an inward-flow direction. The containment shell may be constructed of a material which is vapor permeable in an outward-flow direction.

The present invention may include a protective umbilical cord cover comprising a containment portion being adapted to protect an umbilical cord of an infant and also an attachment mechanism being adapted to releasably attach the containment portion against the infant. The attachment mechanism may comprise an adhesive. The containment portion may comprise a containment shell and the protected air space positioned between the infant and said containment shell, wherein said containment shell may be constructed of a material which is liquid impermeable in an inward-flow direction. The containment shell may be constructed of a material which is vapor permeable in an outward-flow direction.

The present invention may include a protective umbilical cord cover comprising a containment portion being adapted to protect an umbilical cord of an infant and also an attachment mechanism being adapted to releasably attach the containment portion against the infant. The attachment mechanism may comprise a fastening mechanism. The containment portion may comprise a containment shell and the protected air space positioned between the infant and said containment shell. The containment shell may be constructed of a material which is liquid impermeable in an inward-flow direction. The containment shell may be constructed of a material which is vapor permeable in an outward-flow direction.

### BRIEF DESCRIPTION OF THE DRAWINGS

FIG. 1A is a front, elevational view of an exemplary embodiment of an umbilical cord cover being worn by an infant in accordance with the present invention;

FIG. 1B is cross-sectional view of an umbilical cord cover shown in FIG. 1A as sectioned along line A-A;

FIG. 2A is a top view of an exemplary embodiment of an umbilical cord cover in accordance with the present invention;

FIG. 2B is cross-sectional view of an umbilical cord cover shown in FIG. 2A as sectioned along line B-B;

FIG. 3 is cross-sectional view of an umbilical cord cover shown in FIG. 2A as sectioned along line B-B wherein permeability flow directions are shown; and

FIG. 4 shows an apparatus for conducting a desiccant method for measuring moisture vapor transmission rate.

FIG. 5 shows an apparatus for conducting an analytical method for measuring the dynamic fluid transmission value of a material.

### DETAILED DESCRIPTION OF THE INVENTION

The present invention relates to a vapor permeable, substantially liquid impermeable protective cover for the umbilical cord of an infant, the cover being temporarily affixable to the wearer.

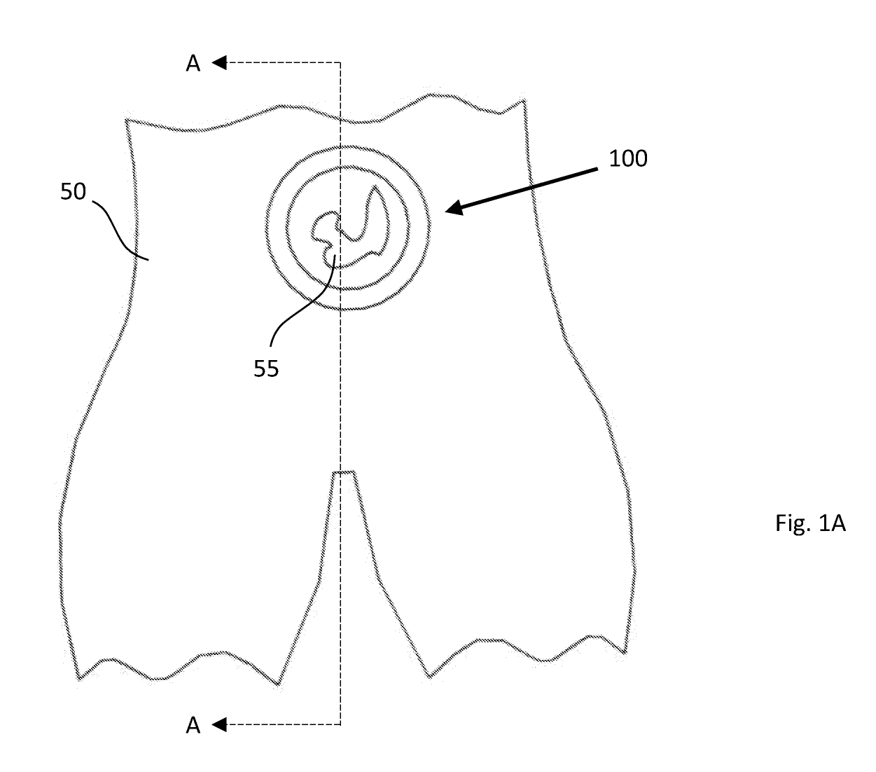

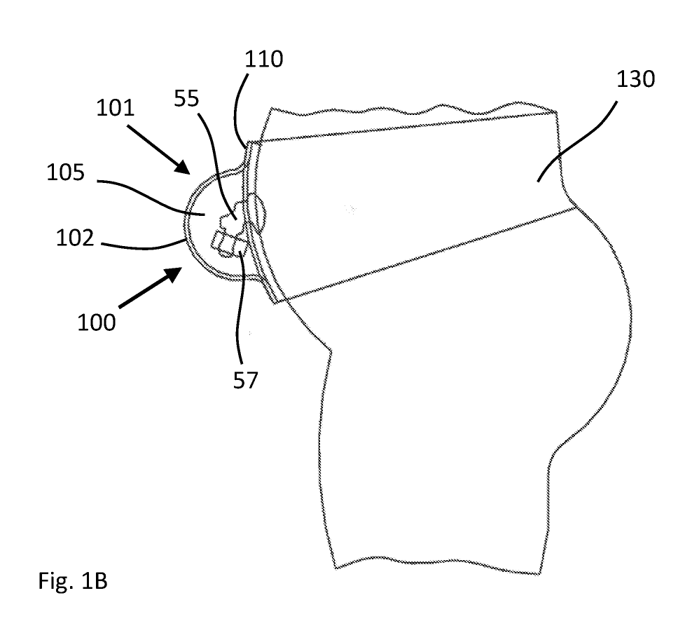

FIG. 1A is a front, elevational view of an exemplary embodiment of a protective umbilical cord cover, 100, being worn by an infant, 50, in accordance with the present invention. The infant, 50, having an umbilical cord, 55, that has not yet dried and fallen off. The umbilical cord cover, 100, provides protection from undesired contact with water, bacteria, clothing and many other items that might harm the umbilical cord, 55, and infant, 50.

FIG. 1B is cross-sectional view of a protective umbilical cord cover, 100, shown in FIG. 1A as sectioned along line A-A. The umbilical cord cover, 100, is shown to provide protection of umbilical cord, 55, and cord clip, 57. The umbilical cord cover, 100, comprises a containment portion comprising a containment shell, 102, that provides an air gap or spacing, 105, between the umbilical cord cover, 100, and umbilical cord, 55. While containment shell, 102, is shown as being substantially domed-shaped, a variety of other shapes may be contemplated by one skilled in the art. In this exemplary embodiment, a belt, 130, is provided as an attachment mechanism for securing the umbilical cord cover, 100, against the body of the infant, 50. While a belt, 130, is shown, other attachment mechanisms may be incorporated so as to releasably attach the umbilical cord cover, 100, to infant, 50. Such attachment mechanisms may include, but are not limited to, adhesive, micro-suction materials and other fastening mechanisms contemplated by one skilled in the art. The embodiment of the protective umbilical cord cover shown in FIG. 1B depicts a flange portion, 110, surrounding the total skin contact area of the cover.

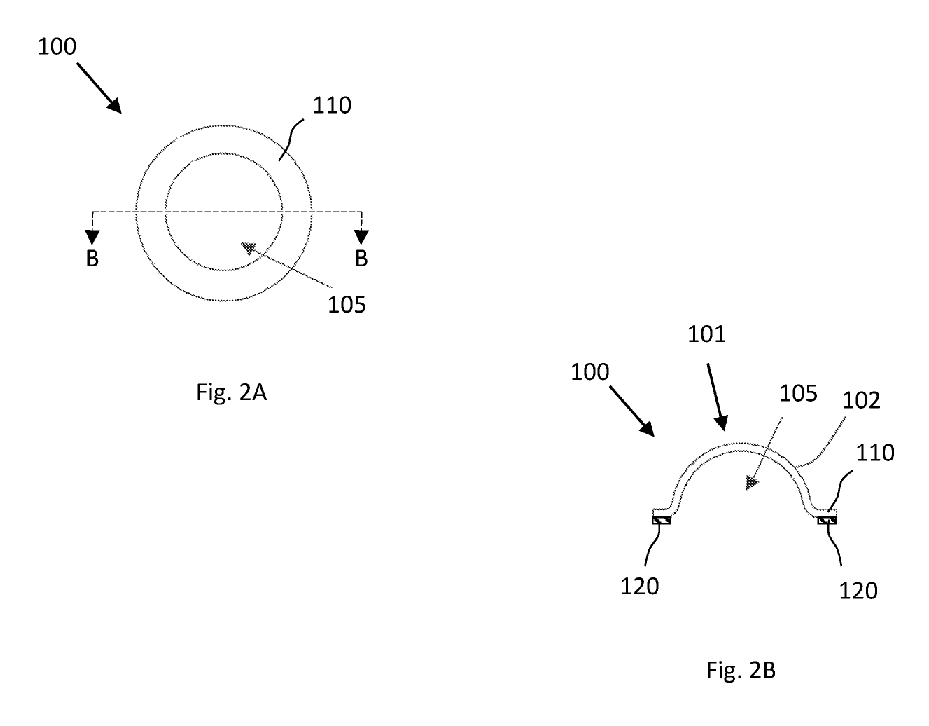

FIG. 2A is a top view of an exemplary embodiment of a protective umbilical cord cover, 100, in accordance with the present invention. The umbilical cord cover, 100, is shown to have a flange portion, 110, that surrounds the containment portion, 105. Flange portion, 110, may substantially surround skin contact area of the containment portion, 105, and provide improved comfort, improved contact and improved protection for umbilical cord, 55.

FIG. 2B is cross-sectional view of an umbilical cord cover, 100, shown in FIG. 2A as sectioned along line B-B. As shown, an alternative attachment mechanism is incorporated to releasably attach umbilical cord cover, 100, to infant, 50, more specifically, in this example adhesive, 120, is positioned on the underneath side of flange portion, 110.

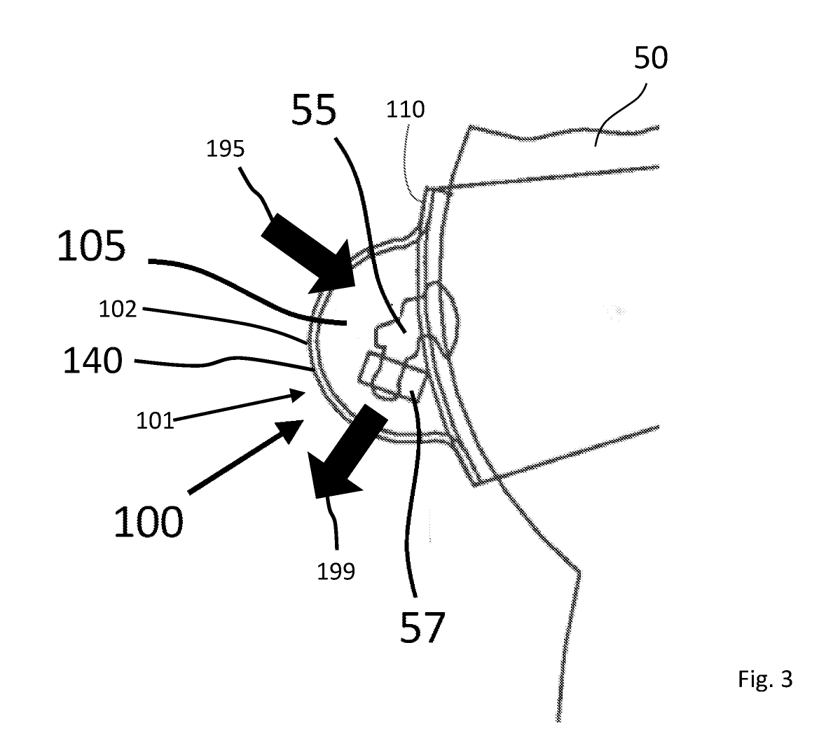

FIG. 3 is cross-sectional view of an umbilical cord cover, 100, shown in FIG. 2A as sectioned along line B-B. In this diagram, containment shell, 102, is shown as being a film. Further in this diagram, arrows are shown to demonstrate “inward-flow direction”, 195, (exterior-to-interior) and “outward-flow direction”, 199, (interior-to-exterior). It may be desirable for containment shell, 102, to have specific permeability characteristics to achieve various product benefits. In one exemplary embodiment, it may be desirable for the containment shell, 102, to be liquid impermeable in an inward-flow direction, 195. Additionally, it may be desirable for the containment shell, 102, to be vapor permeable in an outward-flow direction, 199. In this way, liquid and other harmful materials would be prevented from entering the containment portion and injuring umbilical cord, 55, while also allowing moisture vapors (e.g., perspiration) from infant, 50, to escape containment portion through the containment shell, 102, to allow umbilical cord, 55, to dry.

### A. Containment Portion:

The containment portion, 101, of the protective umbilical cord cover, 100, is the portion of the cover intended to contain and accommodate the umbilical cord of the wearer. The containment portion, 101, may comprise a containment shell, 102, and a protected air space, 105, positioned between the infant, 50, and the containment shell, 102. In order for the containment portion, 101, to be able to accommodate the umbilical cord, it must have some degree of available void-space, 105. The required void-space can be created via deformation of the containment shell, 102, such as by molding, vacuum forming, blow molding or other deformation techniques known in the art. Such structures would have a three dimensional shape following deformation. The containment shell, 102, of the containment portion, 101, of the present invention may comprise a rounded dome to form a void-space, 105, to accommodate the umbilical cord, 55, although other shapes may be contemplated for the void-space shape.

Alternatively, the containment shell, 102, may comprise a low modulus-extensible material which when applied to the wearer is deformed around the umbilical cord thereby creating a void-space to accommodate the cord. In yet another embodiment, the containment shell may comprise an elastically extensible material, preferably a low modulus elastically extensible material which when applied to the wearer extends to provide the void-space necessary to accommodate the umbilical cord.

The containment shell, 102, may also be constructed of materials that are nonabrasive to the skin and umbilical cord. The materials of the containment shell should not easily adhere to the umbilical cord, either due to bleeding or liquid seepage from the cord. The materials used in the containment shell may also have a relatively smooth surface to prevent a dry, rough cord from becoming entangled with the material, such as when the cord becomes entangled in the clothing. Materials particularly suited for use in the containment shell include the films described earlier in this application as well as some smooth nonwoven materials. The materials of the containment shell may also be substantially liquid impermeable and also highly vapor permeable as described throughout this application.

### B. Containment Shell:

The containment shell, 102, is the layer of the containment portion, 101, that when in place forms the necessary void-space, 105, to protect the wearer's umbilical cord, 55. The containment shell, 102, may be constructed using a variety of materials that provide or promote breathability (i.e., exchange of air and/or moisture vapor) while delivering a high level of liquid impermeability. Such breathable, vapor permeable materials promote proper drying of the umbilical cord. The vapor permeable materials allow the moisture vapor associated with the TEWL of the infant's skin and drying of the umbilical cord to be released through the material of the containment shell, such as by diffusion.

Certain polymeric films comprise micropores in the film which make the films breathable (i.e., moisture vapor permeable) these types of polymeric films are referred to as microporous films. In microporous films, moisture is transported through the films by way of small gaps or holes in the film. One notable microporous film composite is made from polytetrafluoroethylene that is adhered to a textile material with an adhesive, as disclosed in British Patent Application No. 2,024,100. Microporous films adhesively bonded to textile substrates have been used in a variety of apparel products, including absorbent articles, as disclosed in PCT Patent Publication Nos. WO 95/116562 and WO 96/39031.

Laminates of a microporous film and a fibrous textile substrate have also been produced. Both the film and the textile substrate are vapor permeable. The textile substrate provides a more clothlike surface which is more comfortable for the wearer than the film layer. Such microporous film laminates have been used as the backsheet, outer cover, of disposable diapers and can be used as a portion of the protective cover of the present invention. These materials allow vapor to pass through the laminate while being substantially impervious to liquids, even when the material comes into direct contact with liquid.

Some types of microporous films can permit transmission of bacteria, viruses, and other microbes through pores in the film. Microbial adsorbents have been added to some microporous films in an attempt to capture microbes passing through such films, as disclosed in PCT Patent Publication No. WO 96/39031. However, it is difficult to distribute microbial adsorbents throughout a microporous film in a manner that will adsorb all microbes seeping through the holes in the film. These materials can, however, provide an effective umbilical cord cover under a variety of circumstances. The bacterial or viral containment concern when using a protective cord cover made of such anti-microbial breathable materials exists only under extreme circumstances. In instances where bacterial or viral containment is a strong concern, the protective umbilical cord cover can be formed of a nonporous monolithic vapor permeable film layer as described later in this application.

An alternative film to the microporous moisture vapor permeable films can comprise polyether ester block copolymers, like the film disclosed in U.S. Pat. No. 4,493,870. The films comprise materials that are non-porous and therefore substantially impermeable to fluids, but they permit the passage of moisture vapor. U.S. Pat. Nos. 4,725,481; 5,422,172; and 5,445,874 disclose such moisture vapor permeable polyether block copolymer films. The aforementioned patents also disclose that such nonporous films can be attached to a variety of fibrous substrates including polyester, polypropylene and nylon. Bonding methods used to join the polyether block copolymer films to the fibrous substrates include adhesive lamination, thermal lamination and extrusion coating. Adhesive lamination and thermal lamination are generally carried out in a two-step process whereby the film is first formed and is subsequently laminated to the fibrous substrate. With extrusion coating, a melted film is extruded directly onto a fibrous substrate and then passed through a nip while the film is still hot in order to press the film into engagement with the fiber network of the fibrous sheet. Adhesive lamination is the preferred lamination process for combining the nonwoven substrate and the vapor permeable film substrate for use in the present invention.

Typical adhesive lamination is carried out in a post-film formation step. For adhesive lamination to be feasible, the moisture vapor permeable film must have enough structure, tensile strength and tear strength such that the film can be formed, wound onto a roll, and later unwound and handled during the adhesive lamination process.

The nonporous moisture vapor permeable film described herein is preferably substantially free of pinholes or pores, yet still has a relatively high rate of moisture vapor transmission. As used herein, “pinholes” means small holes inadvertently formed in a film either during manufacture or processing of the film, while “pores” means small holes in a film that are intentionally formed in the film in order to make the film porous to air, moisture vapor or liquids.

In a preferred embodiment of the invention, the containment shell comprises a moisture vapor permeable, substantially liquid impermeable film is a polyether block copolymer such as copolymers comprised of block copolyether esters, block copolyether amides, polyurethanes, polyvinyl alcohols, or combinations thereof. The fibrous substrate is preferably comprised of synthetic polymer fibers in a form to which the moisture vapor permeable film can be adhered. The substrate may be a woven or nonwoven structure, but for cost reasons, nonwoven textile structures are preferred for most applications.

For embodiments comprising a film layer, the film layer is preferably moisture vapor permeable, in the outward-flow direction, and substantially liquid impermeable film in the inward-flow direction. The film layer may comprise a microporous film layer or alternatively the film layer may comprise a thermoplastic polymer material that can be extruded as a thin, continuous, nonporous, substantially liquid impermeable film, preferably both types of film layer materials will be moisture vapor permeable. The nonporous moisture vapor permeable film is preferably comprised primarily of a block copolymer, such as a polyether ester copolymer, a polyether amide copolymer, a polyurethane copolymer, polyvinyl alcohol, or a combination thereof. Preferred copolyether ester block copolymers are segmented elastomers having soft polyether segments and hard polyester segments, as disclosed in U.S. Pat. No. 4,739,012 (assigned to DuPont). Suitable polyether ester block copolymers are sold by DuPont under the name Hytrel®. Hytrel® is a registered trademark of DuPont. Suitable copolyether amide copolymers are copolyamides available under the name Pebax® from Atochem Inc. of Glen Rock, N.J., USA. Pebax® is a registered trademark of Elf Atochem, S.A. of Paris, France. Suitable polyurethanes for use in film layer are thermoplastic urethanes available under the name Estane® from The B.F. Goodrich Company of Cleveland, Ohio, USA.

According to another embodiment of the present invention, the film layer may be a moisture vapor permeable, substantially liquid impermeable multiple layer film structure. Such a film may be coextruded with layers comprised of the one or more of the above described moisture vapor permeable film materials described herein. According to another embodiment of the invention, a thin moisture vapor permeable film could be used in conjunction with a microporous film to form a laminate film structure. Such a structure overcomes a number of the drawbacks associated with some microporous films, namely bacteria and liquid seepage and h h moisture impact values, without sacrificing the relatively high MVTR values, often >3,000 g/m/day, obtainable with some microporous films. The moisture vapor permeable films of the containment shell of the present invention can be made compatible with polyolefin nonwoven materials and can also be made compatible with current microporous film compositions, such as those of polyolefin composition. The moisture vapor permeable film layer of the containment shell of the present invention and a microporous film can be joined via adhesive lamination or by direct extrusion coating. The moisture vapor permeable film could be combined with a fibrous substrate in a fashion consistent with the present invention. This fibrous substrate and moisture vapor permeable substantially liquid impermeable film can be joined to a microporous film in a fashion consistent with the present invention, such that the nonwoven fibrous substrate will be bonded to the first side of the moisture vapor permeable, substantially liquid impermeable film layer and the microporous film will be laminated to the opposing side of the film layer.

According to one preferred process for making the containment shell, an adhesive is applied to the surface of the fibrous substrate to which the moisture vapor permeable film is to be attached prior to application of the film. The adhesive is preferably applied to the substrate in a dispersed spray pattern at a basis weight of between 3.2 and 38.7 mg/cm (0.5 and 6 mg/in). It is important that the applied adhesive cover less than 75%, and more preferably less than 50%, and most preferably less than 25%, of the surface of the fibrous substrate so that the film layer coated over the adhesive will be discretely bonded to the fibrous substrate and the adhesive will not significantly reduce the moisture vapor transmission rate of the containment shell.

A preferred adhesive is a pressure sensitive hot melt adhesive such as a linear styrene isoprene styrene (“SIS”) hotmelt adhesive, but it is anticipated that other adhesives, such as polyester of polyamide powdered adhesives, hotmelt adhesives with a compatibilizer such as polyester, polyamide or low residual monomer polyurethanes, other hotmelt adhesives, or other pressure sensitive adhesives could be utilized in making the containment shell of the invention. Preferably the adhesive is applied to the surface of the fibrous sheet by an optional glue applicator 39 just before the polymer melt that will form the moisture vapor permeable film layer is extruded onto the substrate. Applicator may comprise a Series 6000 Melter and CF215 Applicator from the Nordson Corporation of Norcross, Ga. Alternatively, the adhesive may be applied to the fibrous substrate and then covered with a release paper and rolled up for storage and subsequent film lamination in another step. The moisture vapor permeable film can then be extrusion-coated over the adhesive and bonded to the fibrous substrate. With this approach, it is believed that the heat from the film melt is sufficient to soften the adhesive in order to promote bonding.

It is believed that the moisture vapor transmission rate (“MVTR”) of a containment shell material used for the present invention is important in promoting proper drying of the umbilical cord. In order to properly dry the umbilical cord, it has been determined that at least a portion of the umbilical cord cover and preferably the entire umbilical cord cover should have a moisture vapor transmission rate of at least about 500 g/m²/day, as measured by the desiccant MVTR measurement method described in the Test Methods Section of this application. The containment shell material of the present invention preferably delivers an MVTR, as measured by the desiccant method, of at least about 1500 g/m²/day, and more preferably at least about 2500 g/m²/day, and most preferably at least about 3500 g/m²/day.

The containment shell of the present invention exhibits the important property that it is substantially impermeable to liquids in the inward-flow direction, under virtually all conditions that are normally associated with the use of the present invention. The liquid impermeability of the containment shell has been characterized according to a number of tests a dynamic barrier test and a number of microbial barrier tests.

The dynamic fluid impact test demonstrates the ability of the containment shell to resist liquid transmission. Suitable materials for the containment shell of the present invention should exhibit substantially little or no dynamic fluid transmission when subjected to an impact energy of about 1000 joules/m². The containment shell material should exhibit less than 50 g/m², preferably less than 10 g/m² more preferably less than 5 g/m², and most preferably less than 1 g/m². The ability of the containment shell to act as a barrier to liquids also prevents the passage of most odors, bacteria, or viruses through the sheet.

When a microporous film was tested according to a bacteria flux test used for evaluating porous sterile packaging materials (ASTM F 1608-95), the material did not pass this test because bacteria was found to pass through the sheet. On the other hand, the nonporous film layer and nonwoven containment shell of the invention, by being impermeable to air during a one hour air porosity test, satisfies the microbial barrier requirement for impermeable sterile packaging materials, as set forth in ISO standard 11607, section 4.2.3.3. The nonporous film layer and nonwoven containment shell has also been shown to prevent the passage of viruses when tested according to ASTM F1671. ASTM F1671 measures the resistance of materials used in protective clothing to penetration of blood-borne viruses such as the Hepatitis B virus (HBV), the Hepatitis C virus (HCV), and the Human Immunodeficiency Virus (HIV) that causes Acquired Immune Deficiency Syndrome (AIDS). This method measure's passage of the surrogate Phi-X174 bacteriophage, which is similar in size to the HCV virus and smaller than the HBV and HIV viruses, through a sheet material.

The film layer in sheet structures according to the invention may additionally contain conventional additives, such as pigments and fillers (e.g. Ti02, calcium carbonate, silicas, clay, talc) and stabilizers, such as antioxidants and ultraviolet absorbers. These additives are used for a variety of purposes, including reducing the cost of the film layer of the containment shell structure, and altering the morphology of the film layer of the sheet structure. However, such additives have been found to reduce moisture vapor transmission through the sheet structure. It is important to maintain the amount of additive in the film at a level that does not result in a moisture vapor transmission rate for the sheet that falls outside of the range required for a particular application. The film layer may be comprised of between 0.01% and 30% of additive material, and more preferably between 0.5% and 7% of an inert filler material.

### C. Attachment Mechanism:

The umbilical cord cover may comprise a variety of attachment mechanisms. The attachment mechanisms may include a belt, adhesive, micro-suction material and other releasably attachable materials. Preferably the attachment mechanism is selected from the group consisting of a belt, an adhesive, and a fastening mechanism.

(i.) It may be desirable for the attachment mechanism to be an adhesive, 120, for adhering a flange portion of the protective cord cover to the abdomen of the wearer. The adhesive may preferably comprise of a substantially liquid impermeable but moisture vapor permeable material and a removable vapor impermeable sheet material releasably secured on the surface of the adhesive opposite the moisture vapor permeable layer of the flange portion. The adhesive being present in the form of a layer between the outer vapor permeable layer and the vapor impermeable layer. The vapor impermeable layer is intended to minimize containment of the adhesive or drying of a hydrogel or hydrocolloid adhesive layer. The adhesive is intended to establish and maintain contact between the flange portion of the umbilical cord cover and the wearer's abdomen. The adhesive is preferably liquid impermeable but moisture vapor permeable. The adhesive may be used in combination with emollients or pectin barriers to minimize effects of epidermal stripping caused by removal of the umbilical cord cover from the skin.

In a preferred embodiment of the adhesive, the adhesive may comprise of a hydrogel or hydrocolloid adhesive. The adhesive layer of the adhesive may be relatively thin (e.g. on the order of between 1 mil and 10 mils thick). If desired, the adhesive layer may also be made substantially thicker (e.g. on the order of 50 mils). While water-activated hydrogel adhesives are generally preferred, the adhesive may alternatively comprise a water-activated hydrocolloid adhesive.

The adhesive layer may be rendered more permeable to vapor diffusion by rendering it porous. The adhesive layer may be rendered porous by mixing an innocuous chemical blowing agent with the adhesive formulation and subsequently applying heat to form an adhesive melt. By controlling the amount of blowing agent and the temperatures employed, the cell size of the resulting foam may be controlled. Foaming or aeration may also be provided by other means known in the art (e.g. direct injection of a gas, volatile liquid or nucleating agent, frothing and the like). The resulting porous adhesive melt may then be coated onto the hydrogel layer in known manner (e.g. extrusion coating, calendaring).

Examples of useful hydrogels for use as the adhesive may comprise a polymer of 2-acrylamido-2-methylpropane sulfonic acid or a salt thereof which are described, for example in U.S. Pat. No. 4,391,278 or U.S. Pat. No. 4,242,242, or Canadian Pat. Nos. 1,173,114, 1,173,116 or 1,173,115, all assigned to Medtronic, Inc.; such hydrogels are commercially available from Medtronic Inc. under the trademarks “EnerTac” NDO Gel and “EnerTac” HH Gel, etc.

Suitable hydrocolloids materials useful as the adhesive include “Hydroactive” (trademark of E.R. Squibb & Sons for the absorbent/adhesive employed in the aforementioned DuoDERM dressing); and the like.

The adhesive may be of any pressure-sensitive adhesive material which lends itself for use on skin. The adhesive layer may comprise a variety of materials (e.g. rubber, rubber-like synthetic homopolymers, copolymers or block polymers, polyacrylate and copolymerisates thereof, polyurethane, silicone, polyisobutylene, polyvinyl ether and natural or synthetic resins or mixtures of these). The adhesive layer may moreover be a hydrogel adhesive as previously mentioned herein. The adhesive matrix may additionally contain various additives, such as plasticizers, thickeners, alcohols and others, as well as, anti-bacterial agents. Optionally hydrogels or hydrocolloid adhesives may comprise various drugs, such as antiseptics, vitamins or antibiotics.

(ii.) Alternatively, the attachment mechanism may comprise an extensible belt, 130, that encircles the waist of the wearer. The belt may preferably comprise of an opening to accommodate the containment portion of the umbilical cord cover. The belt may comprise natural rubber, lycra or polyurethane elastic materials in combination with nonwoven or film layers. The belt may be preferably extensible, more preferably elastically extensible.

In yet another embodiment of the present invention, the attachment mechanism may include a belt which may comprise a material or materials which is pleated by any of many known methods. Alternatively, all or a portion of the belt may be made of a formed web material or a formed laminate of web materials like those described in U.S. Pat. No. 5,518,801 issued on 21 May 1996 in the name of Chappell et al. This formed web material includes distinct laterally extending regions in which the original material has been altered by embossing or another method of deformation to create a pattern of generally longitudinally oriented alternating ridges and valleys. The formed web material may also include a laterally extending unaltered regions located between the laterally extending altered regions.

Such a formed web material can be laterally extended beyond its original dimension with the application of relatively less force than that required to extend the same material to the same extent when un-deformed. In particular, the application of opposing divergent forces directed generally perpendicular to the ridges and valleys extends such a formed web material along an axis between the opposing forces and generates a resistive contractive force, primarily in the unaltered regions. This resistive force is relatively smaller than the resistive force that is generated by the same material in its unaltered form when extended to the same extent, at least up to an extension at which the ridges and valleys in the altered regions flatten and begin to contribute to the resistive force. Thus, such formed web materials exhibit an extensible behavior resembling that of traditional elastic materials in the range of extensibility that is useful in a belt-like affixing means. In addition, different portions of the belt affixing means may be formed to have different ranges of extensibility and/or to be extensible to a greater or lesser degree when subjected to a given level of opposing tensile forces, i.e., to be relatively more easily or less easily extensible.

(iii.) Alternatively, the attachment mechanism may comprise a fastening mechanism, such as, using Velcro-like materials to attach the containment shell to the inside of the clothes of the wearer. Alternatively, the fastening mechanism may comprise of micro-suction materials that adhere to the skin of the wearer.

### D. Flange Portion:

The protective umbilical cord cover may optionally comprise a flange portion, 110, to provide a surface area that is substantially parallel and conformable to the skin of the wearer. This surface area helps to provide an improved seal between the cover and the skin against moisture, outside contaminants and other undesirable materials. This surface area may also provide an area of adhesion for the containment shell to be worn against the skin of the wearer.

The flange portion, 110, may be made of the same material as the containment portion. Alternatively, the flange portion may comprise a material that is different from that of the containment portion.

In a preferred embodiment, the protective cord cover comprises a vacuum formed liquid impervious, vapor permeable film layer having a containment shell in the form of a hemisphere, a flange portion surrounding the opening of the containment shell and an adhesive comprising a low peel strength hydrogel material. Prior to use, the hydrogel material may be covered by an impervious layer to prevent the hydrogel from drying and thereby losing some of its efficacy.

In an alternative embodiment, the protective cord cover comprises a containment shell made of a low-modulus extensible material, a flange portion and a low peel strength adhesive. The flange portion may have a different thickness, stiffness or extensibility relative to the containment portion, thereby enabling the flange portion to retain its shape. Alternatively, the adhesive may also comprise a stiffening element to help maintain the shape of the flange portion.

E. Protective Umbilical Cord Cover with Umbilical Line

In a specific embodiment of the protective umbilical cord cover of the present disclosure, the cover may be modified to allow fitting of a medical umbilical line through the containment shell.

An umbilical line is a catheter that is inserted into one of the two arteries or the vein of the umbilical cord. Generally the Umbilical Artery Catheter/Umbilical Vein Catheter (“UAC/AVC”) is used in neonatal intensive care units as it provides quick access to the central circulation system of premature infants. UAC/UVC lines can be placed at the time of birth and allow medical staff to quickly infuse fluids, inotropic drugs, and blood if required. Medications, fluid, and blood can be given through this catheter and it allows monitoring of blood gasses and withdrawing of blood samples. One complication of the use of UAC/UVC lines is umbilical infections.

This embodiment of the protective umbilical cord cover further comprises and sealable opening, through which the UAC/UVC line may be run from whichever medical equipment or medical delivery system to the infant's umbilical cord. The line may be sealed in place to the containment shell by any method know to those skilled in the art. The seal may be permanent or temporary, but sufficient to maintain the moisture seal of the containment shell.

### F. Test Methods

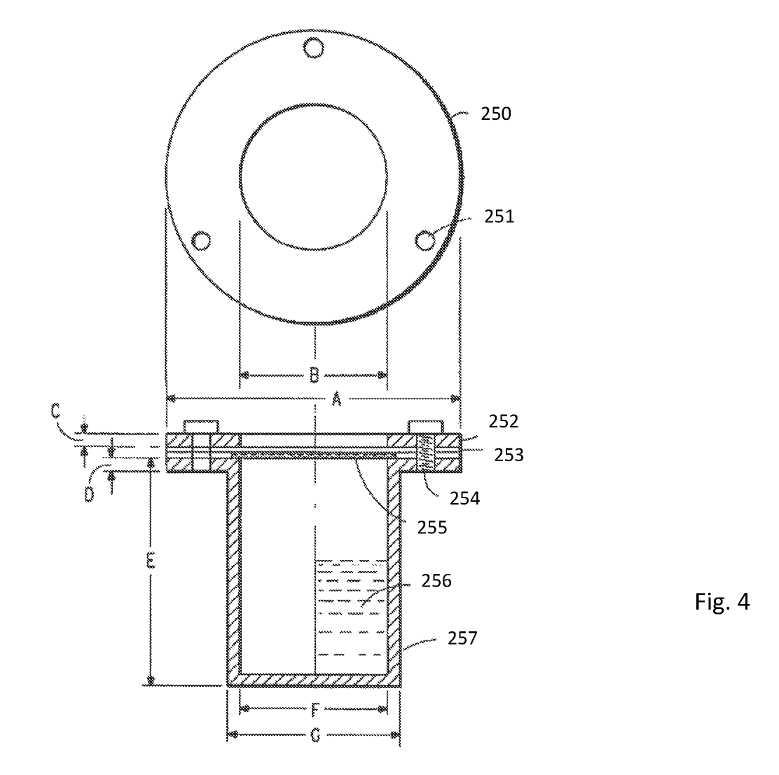

Moisture Vapor Transmission Rate, (MVTR) may be determined by a method that is based in part on ASTM E96, which is hereby incorporated by reference, and is reported in g/m²/day. This method is referred to as the “desiccant method” for measuring moisture vapor transmission rate as set forth below. Briefly summarizing this method, a defined amount of calcium chloride desiccant (CaCl₂) is put into a flanged “cup” like container, see FIG. 4 shown with a partial cutaway. The sample, 255, material is placed on the top of the container, 257, and held securely by a retaining ring, 252, and gasket, 253. The assembly is then weighed and recorded as the initial weight. The assembly is placed in a constant temperature (40° C.+/−3 C) and humidity (75% RH+/−3%) chamber for five (5) hours. The assembly is then removed from the chamber, sealed to prevent further moisture intake, and allowed to equilibrate for at least 30 minutes at the temperature of the room where the balance is located. The amount of moisture absorbed by the CaCl₂, 256, is determined gravimetrically and used to estimate the moisture vapor transmission rate (MVTR) of the sample by weighing the assembly deducting the initial weight from the final assembly weight. The moisture vapor transmission rate (MVTR) is calculated and expressed in g/m²/day using the formula below. Samples are assayed in triplicate. The reported MVTR is the average of the triplicate analyses, rounded to the nearest 100. The significance of differences in MVTR values found for different samples can be estimated based on the standard deviation of the triplicate assays for each sample.

Suitable Analytical Balances for performing the gravimetric measurements include a Mettler AE240 or equivalent (300 g capacity) or a Sartorius 2254S0002 or equivalent (1000 g capacity). A suitable sample holding assembly comprises a cup, 257, and retaining ring, 252, machined from Delrin® (such as that available from McMaster-Carr Catalog #8572K34) with a gasket, 253, made of GC Septum Material (Alltech catalog #6528). The dimensions of the cup, retaining ring and gasket are as follows: the dimensions of the cup are A which corresponds to the retaining ring outer diameter and cup flange diameter is 63 mm, B is 55 mm, C which is the retaining ring thickness is 5 mm, D which is the flange thickness is 6 mm, E is the cup height and the dimension is 55 mm, F corresponds to the inner diameter of the cup and also the diameter of the opening in the retaining ring this dimension is 30 mm, G is the outer diameter of the cup which is 45 mm. The desiccant comprises CaCl₂, 256, for U-tubes, available from Wako Pure Chemical Industries, Ltd., Richmond, Va. Product#030-00525. The plastic food wrap comprises Saran Wrap, available from Dow Chemical Company, or equivalent. A suitable environmental chamber is available from Electro-Tech Systems, Inc, ETS, model 506A or equivalent. The temperature controller is ETS model 513A or equivalent, the humidity controller is ETS model 5I4 or equivalent, the heating unit is a Marley Electric Heating Model 25I2WC (400 watts) or equivalent, the humidifier is ETS model 5612B or equivalent.

The CaCl₂ can be used directly from a sealed bottle as long as the size of the lumps is such that they do not pass through a No. 10 sieve. Usually the top two-thirds of the bottle does not have to be sieved. However, the bottom third contains fines that should be removed by sieving. The CaCl₂ can be used from a closed container without drying. It can be dried at 200° C. for 4 hours if required.

Representative samples should be obtained from the materials to be tested. Ideally, these samples should be taken from different areas of the material so as to represent any variations present. Three samples of each material are needed for this analysis.

Samples should be cut into rectangular pieces approximately 1.5″×2.5″. If the samples are not uniform, clearly mark the area for which breathability is to be evaluated. If the samples are not bidirectional, clearly mark the side that is to be exposed to high humidity. For samples used in diapers and cat menials, this is usually the side that contacts the absorbent layer of the article or the wearer in the case of garments.

To begin a test session, (I) weigh approximately 15 grams of CaCl₂, 256, and place in the MVTR cup 257. Gently tap the cup, 257, about 10 times on the bench top to distribute and lightly pack the CaCl₂. The CaCl₂, 256, should be level and about 1 cm from the top of the cup, 257. Adjust the amount of CaCl₂ until the 1 cm distance is achieved. Then (2) place the sample, 255, with the high humidity side up (if required), over the opening in the top of the cup, 257. Make sure that the sample overlaps the opening so that a good seal will be obtained. Next, (3) place the gasket material, 253, and the retaining ring, 252, on the top of the cup, aligning the screw holes and checking to make sure that the sample has not moved. Tighten the screws, 254, to securely fasten the retaining ring, 252, and seal the sample to the top of the cup. Care should be taken to not over tighten the screws, 254, as this leads to distortion of some samples. If distortion of the sample occurs, loosen the screws, 254, and tighten again. Then (4) weigh the MVTR cup assembled in step 3. Record this weight as the initial weight. This process should be conducted in a relatively short time per cup, <2 minutes.

After weighing the assembly, (5) place the sample in the CT/CH chamber for 5.0 hours (to the nearest minute). When the time has elapsed, (6) remove the sample from the CT/CH chamber, tightly cover it with plastic wrap secured by a rubber band. Record the time of sample removal to within the nearest minute. Allow samples to equilibrate for at least 30 minutes at the temperature of the room where the balance is located. After equilibration, (7) remove the plastic wrap and rubber band and weigh the cup. Record this weight as the final weight. The MVTR is then calculated in units of g/m²/day using the formula:

$$MVTR = \frac{( final weight - initial weight ) \times 24.0}{area of sample in meters \times 5.0 ( time in chamber )}$$

where: 24 is used to convert the data to the 24 hour basis
the area of sample is equal to the open area of the mouth of the cup; and 5.0 is the duration of the test in hours.
   Calculate the average MVTR for each set of triplicate. Round the average MVTR for each sample set to the nearest 100. Report this value as the MVTR for the sample of material.

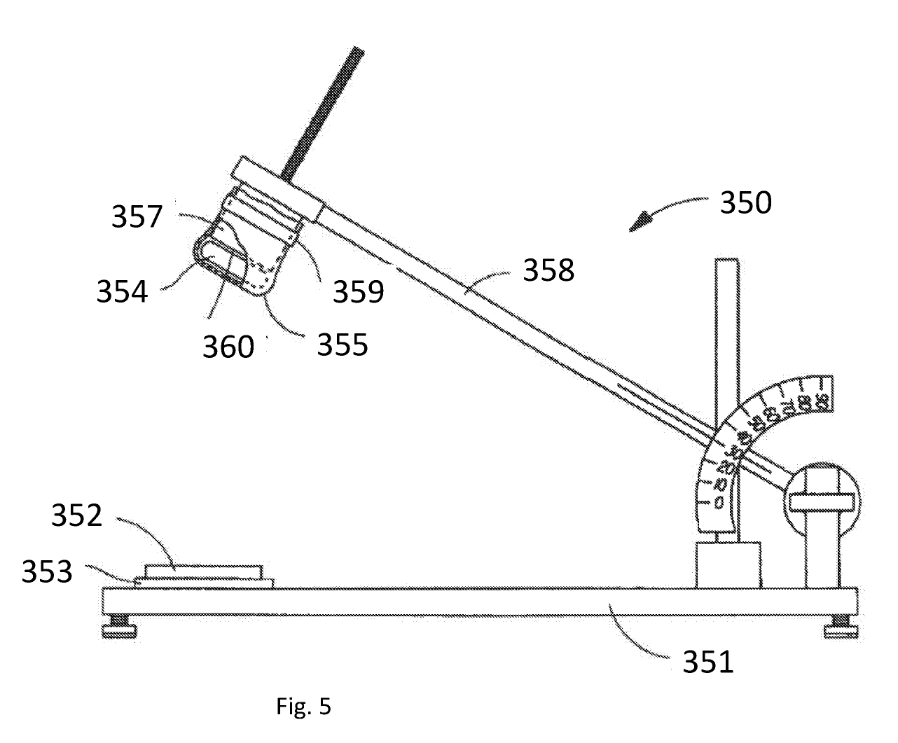

Dynamic Fluid Transmission is measured with the apparatus, 350, shown in FIG. 5. According to this test, an absorption material, 352, weighed to the nearest 0.0001 gram is placed directly on top of the energy absorbing impact pad, 353. The absorption material, 352, may comprise a No. 2 filter paper available from Whatman Laboratory Division, Distributed by VWR Scientific of Cleveland, Ohio The absorption material should be able to absorb and retain the distilled water which passes through the sheet material being tested. The energy absorbing impact pad, 353, is a carbon black filled cross linked rubber foam. The 5 inch by 5 inch square impact pad has a density of 0.1132 g/cm³ and a thickness of 0.3125 inches. The impact pad, 353, has a Durometer Value of A/30/15 according to ASTM 2240-91. A circular absorbent core material, 354, measuring 0.0572 meters (2.25 inches) in diameter is weighed. The absorbent core material may preferably comprise of an individualized, crosslinked wood pulp cellulosic fibers as described in U.S. Pat. No. 5,137,537 issued to Herron et al. on Aug. 11, 1992. The absorbent core material should be able to hold a sufficient amount of distilled water, e.g., at least about ten times its dry weight. The absorbent core has a basis weight of about 228 g/m. The absorbent core material is then is loaded with distilled water to about 5 times its dry weight. In circumstances where the aforementioned crosslinked wood pulp cellulosic fibers are not available, saturated paper towel can be used in place of the wood pulp. When saturated paper towels are used the wet weight of the paper towel should be at least 20 grams to provide an adequate amount of liquid for the dynamic fluid transmission test.

A section of the backsheet material, 355, to be tested, is placed face down with the outside surface on a clean and dry tabletop. The loaded core material, 354, is placed directly in the center of the backsheet material, 355. The backsheet/core arrangement is then secured to the impact portion, 357, of the impact arm, 358, with a rubber band, 359. The backsheet/core arrangement is positioned such that the core, 354, is adjacent the bottom surface, 360, of the impact portion, 357. The impact arm, 358, is raised to a desired impact angle to provide the desired impact energy. The impact arm, 358, is dropped and the impact arm, 358, is then immediately (about 1 second after impact) raised and the filter paper, 352, is removed and placed on a digital scale. The mass of the wet filter paper is then recorded at the one minute mark. The dynamic fluid transmission value (DFTV) is calculated and expressed in g/m using the following formula:

$$DFTV = \frac{\begin{matrix}mass of the wet filter paper ( grams ) - \\ mass of the dry filter paper ( grams )\end{matrix}}{impact area ( m 2 )}$$

The impact area, expressed in m2, is the area of the bottom surface, 360, of the impact portion, 357. The impact area is 0.00317 m2. The absorbent core material, 354, should have an area slightly larger than that of the impact area of the bottom surface, 360.

Bacterial Barrier for Sterile Packaging is measured according to ISO 11607 which states under section 4.2.3.2 that a material that is impermeable to air for one hour (according to an air porosity test) satisfies the standard's microbial barrier requirements. With regard to porous materials, section 4.2.3.3 of ISO 11607 states that there is no universally applicable method of demonstrating microbial barrier properties in porous materials, but notes that the microbial barrier properties of porous materials is typically conducted by challenging samples with an aerosol of bacterial spores or particulates under a set of test conditions which specify the flowrate through the material, microbial challenge to the sample, and duration of the test. One such recognized test is ASTM F 1608-95.

Viral Barrier properties were also measured according to ASTM F1671. ASTM F1671 is a standard test method for measuring the resistance of materials used in protective clothing to penetration by blood-borne pathogens. According to this method, three samples of a sheet material being tested are challenged with 10⁸ Phi-X174 bacteriophage, similar in size to the Hepatitis C virus (0.028 microns) and with a surface tension adjusted to 0.042 N/m, at a pressure differential of 2 psi (13.8 kPa) for a 24 hour period. Penetration of the sample by viable viruses is determined using an assay procedure. The test results are reported in units of Plaque Forming Units per milliliter PFU/ml. A sample fails if any viral penetration is detected through any of the samples. A positive and negative control is run with each sample set. The positive control was a microporous membrane with a pore size of 0.04 microns which passed 600 PFU/ml. The negative control is a sheet of Mylar® film, which passed 0 PFU/ml.

What is claimed is:

1. A protective umbilical cord cover comprising:
a containment portion being adapted to protect an umbilical cord of an infant; and
an attachment mechanism being adapted to releasably attach the containment portion against the infant.

2. The protective umbilical cord cover of claim 1 wherein the containment portion comprises a containment shell and the protected air space positioned between the infant and said containment shell.

3. The protective umbilical cord cover of claim 2 wherein said containment shell is constructed of a material which is liquid impermeable in an inward-flow direction.

4. The protective umbilical cord cover of claim 2 wherein said containment shell is constructed of a material which is vapor permeable in an outward-flow direction.

5. The protective umbilical cord cover of claim 3 wherein said containment shell is constructed of a material which is vapor permeable in an outward-flow direction.

6. The protective umbilical cord cover of claim 4 wherein said containment shell delivers an MVTR, as measured by the desiccant method, of at least about 1500 g/m²/day.

7. The protective umbilical cord cover of claim 5 wherein said containment shell delivers an MVTR, as measured by the desiccant method, of at least about 1500 g/m²/day.

8. The protective umbilical cord cover of claim 1 wherein said attachment mechanism comprises a belt.

9. The protective umbilical cord cover of claim 8 wherein said belt is made of an extensible material.

10. The protective umbilical cord cover of claim 8 wherein said belt is made of an elastically extensible material.

11. The protective umbilical cord cover of claim 1 wherein said attachment mechanism comprises an adhesive.

12. The protective umbilical cord cover of claim 11 wherein said adhesive comprises a hydrogel adhesive.

13. The protective umbilical cord cover of claim 11 wherein said adhesive comprises a hydrocolloid adhesive.

14. The protective umbilical cord cover of claim 1 wherein said attachment mechanism comprises a fastening mechanism.

15. The protective umbilical cord cover of claim 14 wherein said fastening mechanism comprises velcro.

16. The protective umbilical cord cover of claim 15 wherein said fastening mechanism comprises micro-suction.

17. The protective umbilical cord cover of claim 1 further comprising a flange portion that substantially surrounds said containment portion and provides greater surface area for contact with the wearer's skin.

18. The protective umbilical cord cover of claim 17 wherein said flange portion provides an underneath surface for an adhesive to be applied.

19. The protective umbilical cord cover of claim 17 wherein said flange portion comprises a moisture vapor permeable material.

20. The protective umbilical cord cover of claim 1 said containment shell comprises an extensible material.

21. A protective umbilical cord cover comprising:
a containment portion being adapted to protect an umbilical cord of an infant;
an attachment mechanism being adapted to releasably attach the containment portion against the infant, wherein said attachment mechanism is selected from the groups consisting of a belt, an adhesive, and a fastening mechanism;
wherein the containment portion comprises a containment shell and the protected air space positioned between the infant and said containment shell, wherein said containment shell is constructed of a material which is liquid impermeable in an inward-flow direction.

22. The protective umbilical cord cover of claim 21 wherein said containment shell is constructed of a material which is vapor permeable in an outward-flow direction.
